(19) United States
(12) Patent Application Publication
(10) Pub. No.: US 2009/0252758 A1
(43) Pub. Date: Oct. 8, 2009

(54) NUTRITIONAL SUPPLEMENT FOR THE PREVENTION OF CARDIOVASCULAR DISEASE, ALZHEIMER'S DISEASE, DIABETES, AND REGULATION AND REDUCTION OF BLOOD SUGAR AND INSULIN RESISTANCE

(76) Inventors: Mohammad A. Mazed, Yorba Linda, CA (US); Sayeeda Mazed, Jharkhand (IN)

Correspondence Address: Haynes and Boone, LLP;IP Section, 2323 Victory Avenue, SUITE 700, Dallas, TX, 75219, US

(21) Appl. No.: 12/169,523

(22) Filed: Jul. 8, 2008

Related U.S. Application Data
(60) Provisional application No. 61/043,059, filed on Apr. 7, 2008.

(51) Int. Cl. A61K 36/02; A61K 36/82; A61K 36/54; A61K 36/886; A61K 36/45; A61P 9/00; A61P 25/28; A61P 3/10; A61K 36/906

(52) U.S. Cl. 424/195.17; 424/729; 424/739; 424/744; 424/732; 424/756

(57) ABSTRACT

A mixture is formulated that is capable of improving a person's well being, lowering the risks of cardiovascular and/or Alzheimer's diseases and/or lowering blood sugar using natural and synthetic ingredients. A mixture of the present invention may be used as a food, a drink, a supplement or potentially a drug. Numerous ratios of ingredients may be formulated to balance flow (viscosity), sediment/uniformity, color, taste, flavor and aroma. Moreover, a method of making certain ingredients soluble in water and/or oil by micro-emulsion and a method encapsulating key ingredient(s) in nano-vessel are described herein.

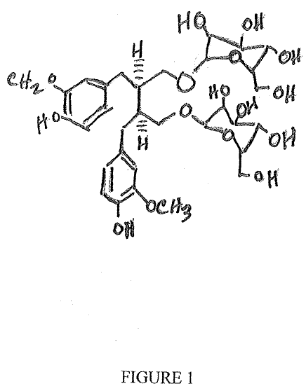

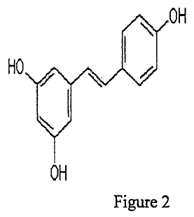

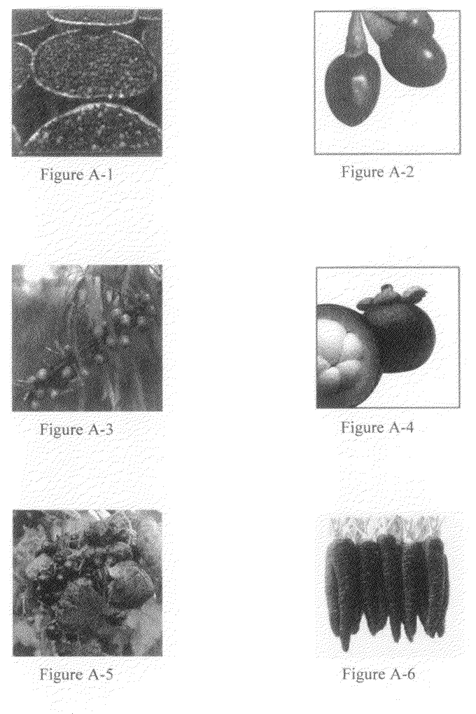

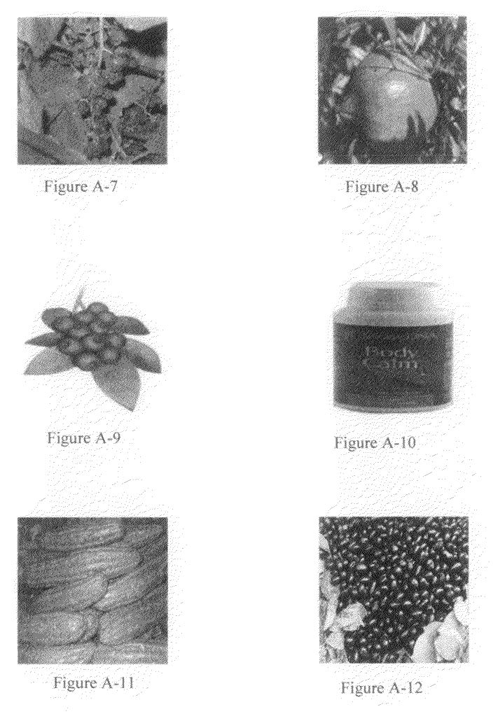

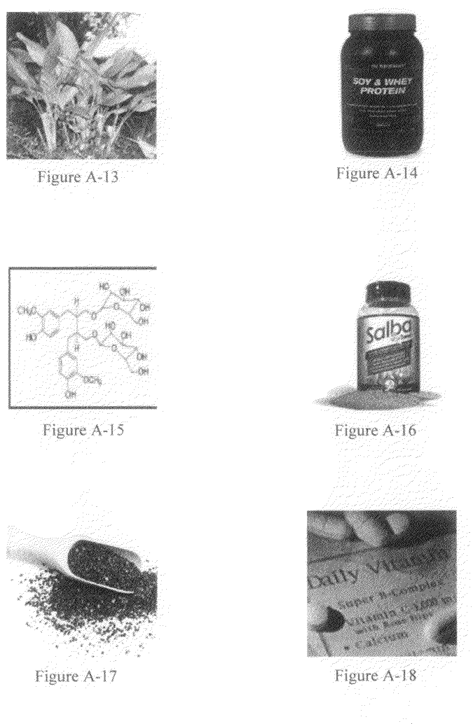

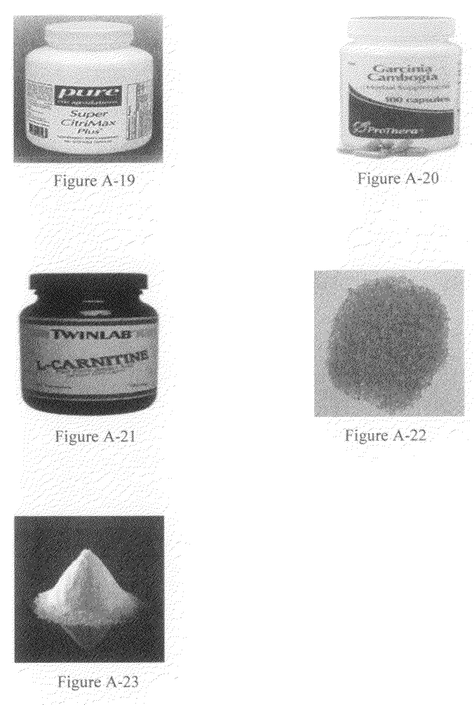

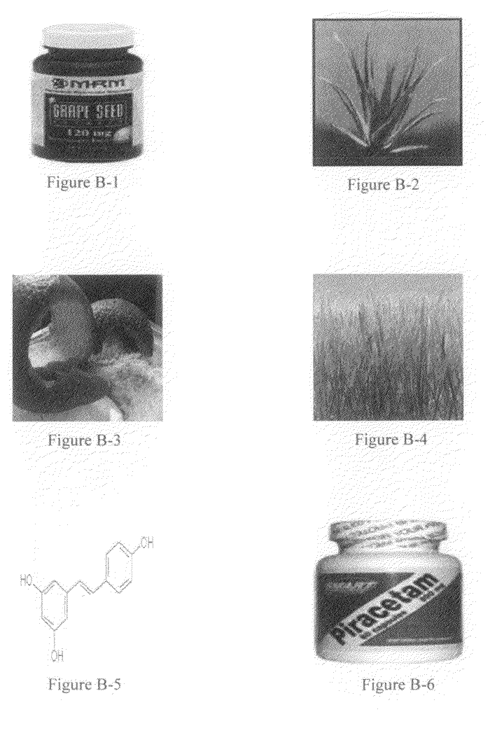

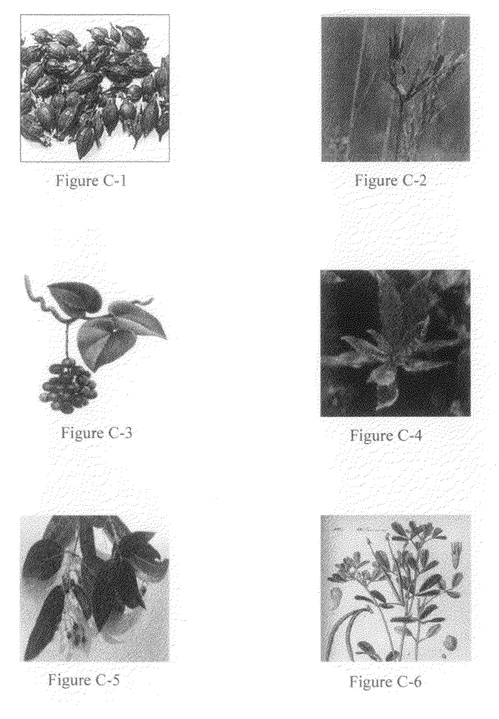

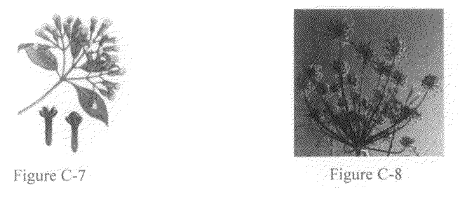

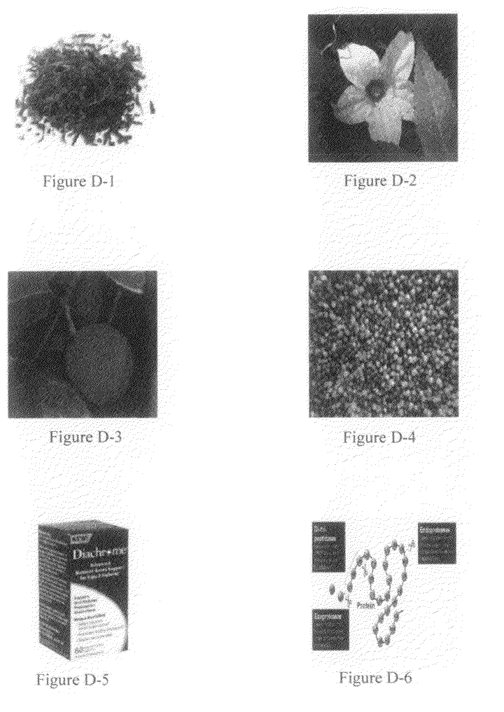

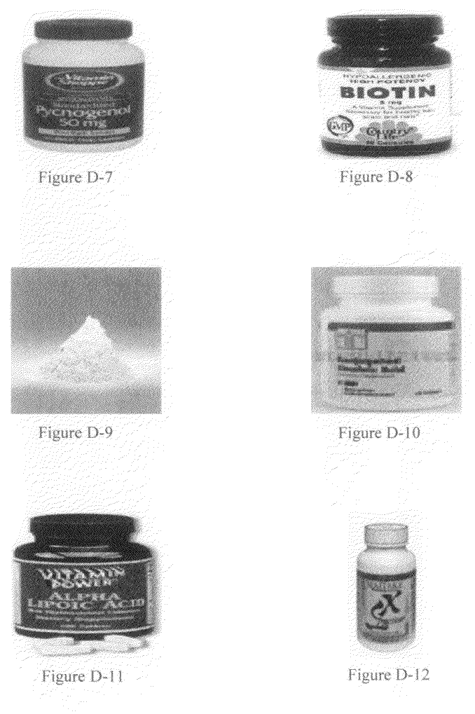

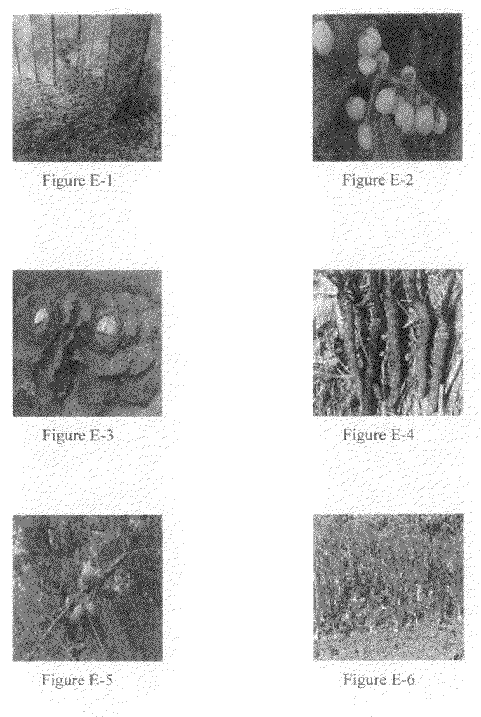

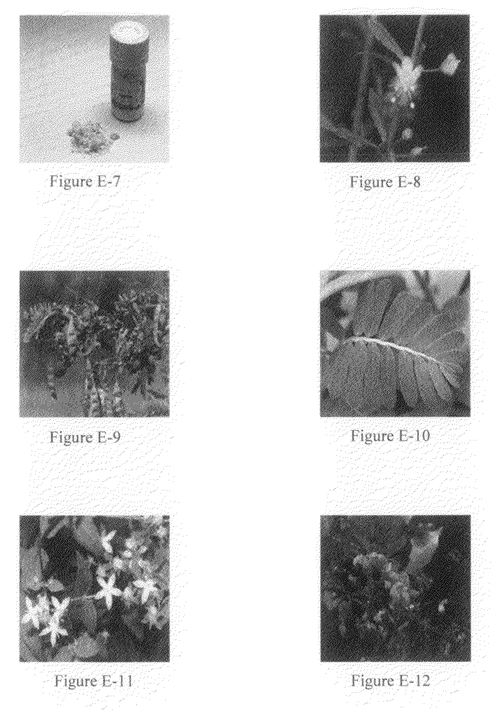

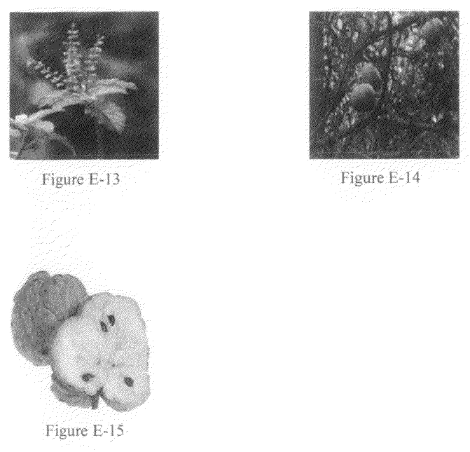

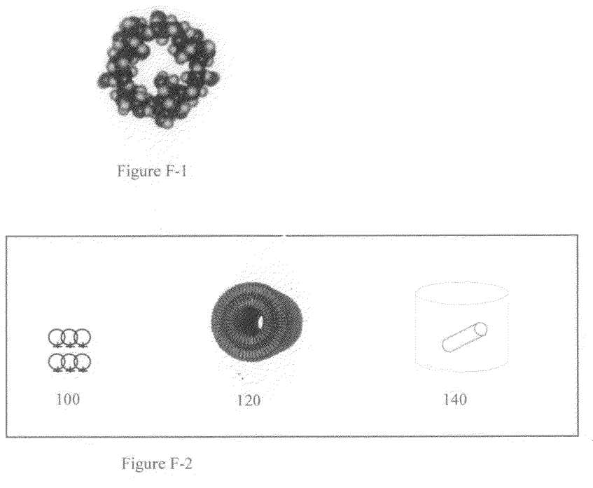

### CROSS REFERENCE OF RELATED APPLICATIONS

The present application is related to and claims priority to U.S. Provisional Patent Application, entitled “A Nutritional Supplement to Aid in the Regulation and Reduction of Blood Sugar” which was filed on Apr. 7, 2008, and assigned Ser. No. 61/043,059. The U.S. Provisional Patent Application 61/043,059 is hereby incorporated by reference in its entirety.

### BACKGROUND OF THE INVENTION

1. Field of Invention

The present invention relates to nutragenomics.

2. Discussion of the Related Art

Many studies suggest that blood cholesterol levels may be an important risk factor for Alzheimer's disease. If blood flow is restricted because of a buildup of plaque or clots in the blood vessels, or if blood becomes too viscous for smooth flow, less oxygen gets to the brain cells and fewer waste residues leave the brain cells. Evidence also suggests a link between cardiovascular-related conditions and dementia. One of the most intriguing recent discoveries is that many risk factors for cardiovascular, Type-1 and Type-2 diabetic diseases are also risk factors for Alzheimer's disease.

Diabetes is closely linked to, in the early stage, metabolic syndrome, and on the later stage, obesity—hence, a new quasi-medical term; diabesity. Because of diabesity, there are macrophages in fat tissues. These macrophages produce “cytokine” molecules. These “cytokine” molecules can cause inflammation in heart and islets of the pancreas hence increasing the insulin resistance in muscle and liver tissues. Diabetes is a disease condition where the body does not produce enough or properly use insulin, a hormone needed to convert glucose, carbohydrates and others into energy needed for daily life. Diabetes is marked by high levels of blood glucose resulting from defects in insulin production and/or insulin action. As the need for insulin rises, the pancreas gradually loses its ability to produce it. Diabetes can lead to serious complications, from high blood pressure to kidney disease, stroke and premature death, but people with diabetes can take steps to control the disease and lower the risk of complications.

Currently, there are approximately 250 million people worldwide living with Type-1 and Type-2 diabetes. The primary causes of diabetes are (1) genetic make-up (2) sedentary lifestyles and (3) dietary habits.

Presently, the known remedies for Type-2 diabetes are: (1) weight/fat reduction, (2) appropriate food, drink and nutritional supplement consumption, (3) stress reduction and (4) smoking cessation.

Bioactive compounds (extra-nutritional constituents) are occurring in small quantities in seeds, vegetables, fruits and legumes. Seeds, vegetables, fruits and legumes are rich in fiber, flavonoids, anthocyanidins, polyphenols and unsaturated fatty acids and low in saturated fat, transfat and cholesterol. There is epidemiologic evidence demonstrating a protective role of these bioactive compounds in seeds, vegetables, fruits and legumes to prevent and/or delay cardiovascular, Type-2 diabetes and/or Alzheimer's diseases.

### SUMMARY OF THE INVENTION

A liquid mixture is formulated to improve a person's well being, with possible implications to lowering the risks of cardiovascular and/or Alzheimer's diseases and/or lowering blood sugar using natural ingredients. A mixture of the present invention may be used as a food, a drink, a supplement or potentially a drug. Numerous ratios may be formulated to balance flow (viscosity), sediment/uniformity, color, taste, flavor and aroma.

The present invention is better understood upon consideration of the examples below in conjunction with the drawings.

### BRIEF DESCRIPTION OF THE DRAWINGS

FIG. A-1 shows examples of Acai Berries (i.e., Euterpe oleracea).

FIG. A-2 shows examples of Goji or Wolfberries (i.e., Lycium barbarum).

FIG. A-3 shows examples of Seaberries (i.e., Hippophae rhamnoides).

FIG. A-4 shows examples of Mangosteens (i.e., Garcinia mangostana)

FIG. A-5 shows examples of Black currants (i.e., Ribes spp).

FIG. A-6 shows examples of Black carrot (i.e., Daucus carota).

FIG. A-7 shows examples of Grapes (i.e., Vitis vinifera, Vitis labrusca, Vitis rotundifolia).

FIG. A-8 shows an example of a Pomegranate (i.e., Punica granatum).

FIG. A-9 shows examples of Blueberries (i.e., Vaccinium spp.—Vaccinium angustifolium, Vaccinium boreale, Vaccinium caesariense).

FIG. A-10 shows a bottle of commercially available Tart Cherry (i.e., Prunus cerasus).

FIG. A-11 shows examples of Bitter Melons (i.e., Momordica charantia (Synonyms: Momordica chinensis, Momordica elegans, Momordica indica, Momordica. operculata, Momordica sinensis, Sicyos fauriei).

FIG. A-12 shows examples of Jamuns (i.e, Syzygium cumini).

FIG. A-13 shows examples of Tumeric Plants (i.e., Curcuma longa).

FIG. A-14 shows a bottle of commercially available Whey, Soy or Rapeseed Protein.

FIG. A-15 shows a molecule of Secoisolariciresinol Diglycoside (SDG) lignans.

FIG. A-16 shows a bottle of commercially available Salba Extract (i.e., Salvia hispanica).

FIG. A-17 shows examples of sprouted Flaxseed (i.e., Linum usitatissimum).

FIG. A-18 shows examples of commercially available Micro-nutrients pills.

FIG. A-19 shows a bottle of commercially available Super CitriMax®.

FIG. A-20 shows a bottle of commercially available Hydroxy Citric Acid (HCA).

FIG. A-21 shows a bottle of commercially available L-Carnitine.

FIG. A-22 shows an example of Fenugreek Gum.

FIG. A-23 shows an example of commercially available Erythritol.

FIG. B-1 shows a bottle of commercially available Grape seed extract (i.e., Vitis vinifera, V. rotundifolia, V. labrusca).

FIG. B-2 shows an example of an Aloe Vera plant (i.e., Aloe vera).

FIG. B-3 shows an example of an Orange peel (Citrus sinensis).

FIG. B-4 shows an example of Wheat Grass (i.e., Triticum aestivum).

FIG. B-5 shows a molecule of Resveratrol or resvida.

FIG. B-6 shows a bottle of commercially available Piracetam®.

FIG. C-1 shows an example of Gardenia (i.e., Gardenia jasminoides).

FIG. C-2 shows an example of an Indian kino tree (i.e., Pterocarpus Marsupium).

FIG. C-3 shows an example of a Guduchi plant (i.e., Tinospora cordifolia).

FIG. C-4 shows an example of a Gurmar or Periploca of the Woods plant (i.e., Gymnema sylvestre).

FIG. C-5 shows an example of a Cinnamon plant (i.e., Cinnamomum zeylanicum, Cinnamomum cassia blume).

FIG. C-6 shows an example of a Fenugreek plant (i.e., Trigonella foenum-graecum).

FIG. C-7 shows an example of a Clove plant (i.e., Syzygium aromaticum).

FIG. C-8 shows an example of a Fennel plant (i.e., Foeniculum vulgare).

FIG. D-1 shows Decaffeinated Green tea (i.e., Camellia sinensis)

FIG. D-2 shows an example of Ivy Gourd flower (i.e., Coccinia indica).

FIG. D-3 shows a Salacia fruit (i.e., Salacia oblonga).

FIG. D-4 shows an example of Resistant starch.

FIG. D-5 shows a box of commercially available Diachrome™.

FIG. D-6 shows a box of commercially available InsuVital™.

FIG. D-7 shows a bottle of commercially available Pycnogenol®.

FIG. D-8 shows a bottle of a commercially available Biotin.

FIG. D-9 shows an example of a commercially available Beta-glucan.

FIG. D-10 shows a bottle of commercially available Conjugated Linoleum Acid (CLA).

FIG. D-11 shows a bottle of commercially available Alpha-Lipoic acid (ALA).

FIG. D-12 shows a bottle of commercially available SX-fraction.

FIG. E-1 shows examples of Stevia (i.e., Stevia rebaudiana).

FIG. E-2 shows examples of Loquats (i.e., Eriobotrya japonica).

FIG. E-3 shows an example of a Cabbage (i.e., Brassica oleracea).

FIG. E-4 shows examples of Chongcaoes (i.e., Cordyceps sinensis).

FIG. E-5 shows examples of Indian Gooseberries (i.e., Phyllanthus emblica (Also known as Emblica officinalis)).

FIG. E-6 shows examples of plants of the Onion family (e.g., onion, garlic, scallion and leeks; i.e., Liliaceae Family).

FIG. E-7 shows examples of Guar gum and its extract (i.e., Cyamopsis tetragonolobus).

FIG. E-8 shows a Licorice weed plant (i.e., Scoparia dulcis—Synonyms: Scoparia grandiflora, Scoparia ternata, Capraria dulcis, Gratiola micrantha).

FIG. E-9 shows an example of a Ranawara or Avaram plant (i.e., Cassia auriculata).

FIG. E-10 shows an example of a Phyllanthus plant (i.e., Phyllanthus niruri).

FIG. E-11 shows an example of an Indian gentian plant (i.e., Swertia chirata).

FIG. E-12 shows an example of an Indian beech plant (i.e., Pongamia pinnata).

FIG. E-13 shows an example of a Tulsi plant (i.e., Ocimum sanctum, Ocimum tenuiflorum).

FIG. E-14 shows an example of a Bael fruit (i.e., Aegle marmelos).

FIG. E-15 shows an example of a Custard apple (i.e., Annona reticulate).

FIG. F-1 shows an example of Cyclodextrins.

FIG. F-2 shows an example of encapsulation of an ingredient in a Nano-tube

### DETAILED DESCRIPTION OF THE PREFERRED EMBODIMENTS

Each of the following examples is a liquid mixture that is formulated for and believed to be effective for improving a person's well being, lowering the risks of cardiovascular, and/or Alzheimer's diseases and/or lowering blood sugar using natural and/or synthetic ingredients. Daily usage is recommended.

### EXAMPLE 1

### Daily Mixture A

A mixture including the following ingredients:

| | | |
|---|---|---|
| Acai berry | mL | 29 |
| Goji Berry | mL | 29 |
| Seaberry &/or Mangosteen | mL | 29 |
| Black Currant &/or Black Carrot | mL | 26 |
| Grape: Red Concord/Red Muscadine | mL | 26 |
| Pomegranate | mL | 26 |
| Blueberry | mL | 26 |
| Red Tart Cherry | mL | 26 |
| Bitter Melon &/or Jamun | mL | 26 |
| Curcumin Powder | Mg | 23 |
| Protein(s): Whey &/or Soy &/or Rapeseed | G | 2 |
| SDG &/or Salba &/or Sprouted Flaxseed | G | 1 |
| Vitamin B₃ | Mg | 3 |
| Vitamin B₅ | Mg | 3 |
| Vitamin B₆ | Mg | 1 |
| Vitamin B₁₂ | Mcg | 2 |
| Vitamin D₃ | Mcg | 6 |
| Vitamin K₁ | Mcg | 1 |
| Boron | Mg | 0 |
| Calcium | Mg | 23 |
| Magnesium | Mg | 1 |
| Selenium | Mcg | 2 |
| Vanadium | Mcg | 6 |
| Zinc | Mg | 1 |
| Omega-3 | Mg | 6 |
| Omega-6 | Mg | 17 |
| 0mega-9 | Mg | 17 |
| Coenzyme Q10 | Mg | 1 |
| Super CitriMax | Mg | 6 |
| L-Carnitine | Mg | 2 |
| Fenugreek &/or Xanthan Gum - If Needed | Mg | 11 |
| Optional Erythritol | G | 1 |
| Color | mL | 0.01 |
| Flavor | mL | 0.01 |
| Total Volume | mL | ~240 |

All or some of the components from the “Other - E” group below may be combined with some or all of the components of Mixture A.

Mixture A has a deep maroon-red color and a fruity flavor. Further information regarding the ingredients is provided as follows:

Acai Berry

Scientific Name: Euterpe oleracea (FIG. A-1)

Acai berry is high in antioxidants, vitamins, minerals, and contains omega 6 and 9 fatty acids, which are associated with heart health.

Nutrient analysis results from 100 grams of powder yields 533.9 calories, 52.2 grams carbohydrates, 8.1 grams protein and 32.5 grams total fat. The carbohydrate portion includes 44.2 grams of dietary fiber. Acai contains high levels of the monounsaturated fatty acid oleic acid (56.2% of total fats). It is also rich in palmitic acid (24.1% of total fats, a saturated fat) and the polyunsaturated omega-6 fatty acid linoleic acid (12.5% of total fats). β-sitosterol (beta-sitosterol), a phytosterol that competes with dietary cholesterol for absorption and so may reduce blood cholesterol levels.

Goji/Wolfberry

Scientific Name: Lycium barbarum (FIG. A-2)

Goji helps stimulate the release of gGH—“youth hormone” and helps to reverse the age-related decline of IgA, an essential immune protein. This can result in an improved immune response, memory, healthy metabolism, sexual energy and anti-flammatory enzyme (SOD).

It contains many nutrients and phytochemicals including: 11 essential and 22 trace dietary minerals; 18 amino acids; 6 essential vitamins; 8 polysaccharides and 6 monosaccharides; 5 unsaturated fatty acids, including the essential fatty acids, linoleic acid and alpha-linolenic acid; beta-sitosterol and other phytosterols; 5 carotenoids, including beta-carotene and zeaxanthin, lutein, lycopene and cryptoxanthin, xanthophyll; numerous phenolic pigments.

Seaberry

Scientific Name: Hippophae rhamnoides (FIG. A-3)

Known as Seabuckthorn or Seaberry may be one of the plant world's most nutritious foods. It has extensive nutrient and phytochemical diversity, including one of the highest vitamin C and E contents, extraordinary pulp and seed levels of alpha linolenic, oleic, palmitic, palmitoleic and linoleic fatty acids, carotenoids and phenolics. Beautifully orange-colored Seaberry is rich in various lipids, natural vitamin A, vitamin C, vitamin B1, vitamin B2, vitamin B6, vitamin E, vitamin F, vitamin P, vitamin K, carotenoids, sugars, flavonoids, minerals, organic acids, irreplaceable amino acids, microcells and other bioactive compounds with nutritional and medicinal properties.

Mangosteen

Scientific Name: Garcinia mangostana (FIG. A-4)

Over 40 different Xanthones have been found in the Mangosteen, which are chemically beneficial molecules, each molecule has remarkable properties. These free radical scavenger/fighters stop pain, reduce swelling and inflammation, and help in the body's healing process. The “strong” antioxidants strengthen cell walls, kill bacteria, viruses, fungus, and stop the attack in DNA and even have restorative properties. They help the body run as a complete, interrelated and integrated system. They help the mind think better, adapt better, and give a sense of well-being (anti-depressant). They stop the damage of brain cells so they grow and divide properly and help the cell walls and the neuro-transmitters function properly. They aid with Alzheimer's disease, and all forms of memory loss. They help in the battle against degenerative diseases like cancer, heart disease, diabetes, arthritis, and all forms of brain malfunctions. They help the vision process: cataracts, macular degeneration, glaucoma, and other forms of vision loss may be prevented. By controlling free radicals, antioxidants can make the difference between life and death, as well as influence how fast and how well we age.

Black Currants

Scientific Name: Ribes spp. Botanical family of Saxifragaceae, Genus Ribes (FIG. A-5)

Black Currants has more vitamin C than any other fruit and are the highest in the antioxidant called anthocyanins. The anthocyanins in Black Currants have shown to be beneficial in warding off ailments including heart disease, cancer, Alzheimer's disease, diabetes and high blood pressure.

Black Carrot

Scientific Name: Daucus carota (FIG. A-6)

Black Carrots contain anthocyanins, part of the flavonoid antioxidant family. Flavonoids may be used as anticancer compounds, as well as inhibitors of LDL (the bad) cholesterol. It has anti-bacterial and anti-fungicidal properties.

Grapes

Scientific Name: Vitis vinifera, V. labrusca, V. rotundifolia (FIG. A-7)

North American grapes (Vitis labrusca and Vitis rotundifolia): Varieties include Concord (blue-black in color and large in size), Delaware (pink-red in color with a tender skin) and Niagara (amber colored and less sweet than other varieties). North American varieties feature skins that more easily slip away from their flesh.

European grapes (Vitis vinifera): Varieties include Thompson (seedless and amber-green in color), Emperor (seeded and purple in color) and Champagne/Black Corinth (tiny in size and purple in color). European varieties feature skins that adhere closely to their flesh.

Grapes contain beneficial compounds called flavonoids, which are phytonutrients that give the vibrant purple color to grapes, grape juice and red wine; the stronger the color, the higher the concentration of flavonoids. These flavonoid compounds include quercitin, as well as a second flavonoid-type compound (falling into the chemical category of stilbenes) called resveratrol.

In addition to resveratrol and saponins, grapes contain yet another compound called pterostilbene, powerful antioxidants that are already known to fight cancer and may also help lower cholesterol.

Researchers have found not only an increase in blood antioxidant activity, but also discovered that grape juice protected LDL cholesterol from oxidation, a phenomenon that can turn LDL into an artery-damaging molecule. Although LDL is often called the “bad” form of cholesterol, it becomes harmful when damaged by free radicals or “oxidized.”

Additionally, it was found that phenolic compounds in grape skins inhibit protein tyrosine kinases, a group of enzymes that play a key role in cell regulation. Compounds inhibiting these enzymes that suppress the production of a bad protein causing blood vessels to constrict hence reduce the flow of oxygen to the heart. This bad protein, called endothelin-1, is thought to be a key contributing agent in the development of heart disease.

Concord grapes have the highest and broadest range of polyphenols and the highest overall antioxidant capacity. The main components in purple grape juice were flavan-3-ols, anthocyanins, and hydroxycinnamates, together accounting for 93% of the total phenolic content.

Muscadine grapes (V. rotundifolia) have high levels of antioxidants and they have more functional genes than wine grapes and therefore provides higher levels of ellagic, OPC's, quercetin, anthocyanidins and resveratrol compounds. These compounds make Muscadine Grape seed a potent antioxidant.

Pomegranate

Scientific Name: Punica granatum (FIG. A-8)

It is among a novel category of exotic plant sources called superfruits. Providing 16% of an adult's daily vitamin C requirement per 100 milliliters serving, pomegranate juice is also a good source of the B vitamin, pantothenic acid, potassium and antioxidant polyphenols. The most abundant polyphenols in pomegranate juice are the hydrolyzable tannins called punicalagins shown to have potent free-radical scavenging ability in laboratory studies. Antioxidant punicalagins absorb into the human body after consumption of pomegranate extracts, and an ex-vivo study of human plasma after consumption of a pomegranate extract standardized to punicalagins indicated an average 32% increase in plasma antioxidant capacity.

Furthermore, pomegranate extracts have no sugar, calories, or additives. Many pomegranate extracts are essentially ellagic acid which absorb into the body after parent molecules, punicalagins are hydrolyzed.

In preliminary laboratory research, pomegranate has been found effective in reducing heart disease risk factors, including LDL oxidation, macrophage oxidative status, and foam cell formation, all of which are steps in atherosclerosis and cardiovascular disease. Tannins such as punicalagins have been identified as the primary components responsible for the reduction of oxidative stress which led to these risk factors. Pomegranate has been shown to reduce systolic blood pressure by inhibiting serum angiotensin-converting enzymes. Interim reports released to the public media were that pomegranate juice may slow onset or development of prostate cancer.

Containing polyphenols which inhibit estrogen synthesis, pomegranate seed oil was effective against proliferation of breast cancer cells in vitro. The juice may also have antiviral and antibacterial effects against dental plaque.

Blueberry

Scientific Names: Vaccinium spp.—Vaccinium angustifolium, Vaccinium boreale, Vaccinium caesariense, (FIG. A-9)

Researchers have shown that blueberry anthocyanins, proanthocyanidins, flavonols and tannins inhibit mechanisms of cancer cell development in vitro. It may alleviate the cognitive decline occurring in Alzheimer's disease and other conditions of aging.

Animal studies found that blueberry consumption lowered cholesterol and total blood lipid levels, possibly affecting symptoms of heart disease and stroke and altered glycosaminoglycans, vascular cell components that can influence control of blood pressure.

One cup (145 g) of blueberries provides 31% of the dietary reference intake for vitamin C, 16% for dietary fiber, 20% for manganese and 7% for vitamin E, with a low glycemic load.

Tart Cherry

Scientific Name: Prunus cerasus (FIG. A-10)

Tart cherry powder made from whole tart cherries may help maintain a healthy cardiovascular system and also fight cell oxidative stress (cell damage caused by free radicals). Tart cherries may offer protection against heart disease, Type-2 diabetes and memory loss.

Bitter Melon

Scientific Name: Momordica charantia (Synonyms: Momordica chinensis, Momordica elegans, Momordica indica, Momordica operculata, Momordica sinensis, Sicyosfauriei) (FIG. A-11)

It contains four very promising bioactive compounds. These compounds activate a 25 protein called AMPK, which is well known for regulating fuel metabolism and enabling the glucose uptake processes which are impaired in diabetics. It may be useful for preventing and treating malaria and HIV.

Jamun

Scientific Name: Syzygium cumini (FIG. A-12)

Common Names: Eugenia Jambolana, Plum, Black Plum, Jaman, Jambolan

The fruit has a combination of sweet, mildly sour, astringent flavor and tends to color the tongue purple. The seed is also used to control diabetes, and is a good source of vitamin A and vitamin C. The quantitative determination showed that Syzygium cumini seeds contained 40% of water soluble gummy fibre and 15% of water insoluble neutral detergent fibre (NDF) and found that the soluble gummy fibre isolated from Syzygium cumini seeds significantly lowered blood glucose levels and improved oral glucose tolerance.

Turmeric

Scientific Name: Curcuma longa (FIG. A-13)

Turmeric may be a remedy for Diabetes. It is more effective if taken with an equal amount of Amla powder. Curcumin extract is one of the major antioxidants in turmeric. Through these antioxidants, Turmeric—Curcumin helps maintain a healthy cardiovascular, a healthy neuroprotective system and a healthy digestive/colon system. It may be possible to dissolve Turmeric and/or Curcumin powder in oil and/or emulsifier like Lecithin and then adding water to the “Turmeric or Curcumin powder and oil/emulsifier mixture”—such a mixture may be ready for dissolving with other ingredients. It may be also possible to utilize Tetrahydrocurcuminoid and water soluble Curcumin derivatives.

Moreover, Curcumin and/or its derivatives have poor bioavailability. However, Curcumin may be encapsulated via micro-emulsion method utilizing ethyl oleate, lecithin and Tween80 as the oil phase and surfactants respectively. When the mole ratio of Lecithin to Tween 80 was 0.3, the optimum capacity of oil solubility was reached 10.3% by weight. However, natural Curcumin has low bioavailability and quickly loses its attributes when ingested. Adding black pepper to Curcumin may boost its absorption. Curcumin with a substance found in cruciferous vegetables like cauliflower and/or broccoli can reduce the growth of prostate tumor cells more effectively than when they're used alone.

Out of many variations of Curcumin molecules, it was found that GO-Y030 and GO-Y031 molecules are more potent and bioavailable.

Whey, Soy or Rapeseed Protein (FIG. A-14)

Whey protein helps control blood glucose levels and has been shown to be beneficial for weight management, both of which are often a concern for Type-2 diabetes. University of Kentucky's meta-analysis concluded that soy protein is correlated with significant decreases in serum cholesterol, Low Density Lipoprotein LDL (bad) cholesterol and triglyceride concentrations. However, High Density Lipoprotein HDL (good) cholesterol did not increase. Water soluble Rapeseed (Canola), Soy and Whey protein are good choices for Type-2 diabetes.

Secoisolariciresinol Diglycoside (SDG) Lignans (FIG. A-15)

Scientific studies show that flax lignans support cardiovascular function, prostate health, bone health, healthy cell replication and hormone balance. Recent study suggests the utility of SDG lignans for cardiovascular health, particularly with impaired glycemic control. Secoisolariciresinol diglucoside (SDG) from flaxseed has been shown to be effective in preventing/delaying the development of Type-1 and Type-2 diabetes. The hypoglycemic effect of SDG in Type-2 diabetes has been suggested to be due to its antioxidant activity. Hyperglycemia in Type-2 diabetes could be due to an increase in the expression of phosphoenolpyruvate carboxykinase (PEPCK), a rate-limiting enzyme in the gluconeogenesis in the liver. It is possible that the hypoglycemic effect of SDG in Type-2 diabetes is due to suppression of expression of PEPCK genes. The results suggest that SDG suppresses the expression of PEPCK genes and that the hypoglycemic effect may be due to suppression of PEPCK gene expression.

Salba

Scientific Name: Salvia hispanica (FIG. A-16)

It is a whole grain that is rich in fiber, alpha-linolenic acid (ALA), and minerals.

A serving of Salba has
More heart-healthy, memory boosting Omega 3's than any other natural source with 3,050 mg per serving
More beneficial fiber than any other natural source (even wheat bran) with more than 5000 mg of dietary fiber per serving
Six times more bone-building calcium than whole milk
Fifteen times more magnesium than broccoli

Sprouted Flaxseed

Scientific Name: Linum usitatissimum (FIG. A-17)

Sprouted Flaxseed has many health benefits. Sprouting doubles the antioxidant (ORAC) value of flaxseed. Nutrients such as enzymes, amino acids, and vitamins are substantially increased and become more bioavailable, allowing for better absorption. The “anti-nutrients” such as phytic acid, enzyme inhibitors and insoluble fibers are decreased.

Micro-Nutrients (FIG. A-18)

Vitamin B₃ (NADH), Vitamin B₅, Vitamin B₆, Vitamin B₁₂, Vitamin D₃, Vitamin E, Vitamin K₁, Calcium, Boron, Magnesium, Selenium, Vanadium, Zinc, Omega 3, Omega 6, Omega 9 and Coenzyme Q₁₀. Moreover, Vitamin B₃ (NADH) may have moderate benefits against depression, Parkinson's and Alzheimer's diseases. Furthermore, Vitamin A and Vitamin C may also be added.

Super CitriMax® (FIG. A-19)

Super CitriMax® curbs appetite, burns fat and results in significant weight loss without side effects; it also promotes healthy blood lipid levels and reduces Body Mass Index, an indicator of healthy body weight. It has been shown to increase levels of serum serotonin, a neurotransmitter involved in mood, sleep and appetite control, which may help address many of the emotional issues overweight people face, including binge-eating and depression.

Hydroxy Citric Acid (HCA) (FIG. A-20)

Garcinia cambogia is a small fruit that resembles a miniature pumpkin. The extract from its fruit and rind is popular in many natural weight loss products. The extract is hydroxycitric acid (HCA), claimed to suppress appetite and enhance fat-burning.

The theory behind that HCA inhibits an enzyme called citrate lyase that helps turn excess carbohydrates into fat. By inhibiting this enzyme, it is believed the body instead boosts carbohydrate oxidation, or simply put, burns the extra carbs. In extensive animal studies, Garcinia cambogia was found to reduce food intake by suppressing appetite, as well as to decrease body fat.

L-Carnitine (FIG. A-21)

Two studies found that supplementing aged rats with either L-Carnitine or alpha-lipoic acid (ALA), improved mitochondrial energy metabolism, decreased oxidative stress, and improved memory.

Fenugreek Gum (FIG. A-22)

It has been confirmed by animal experiments and clinical tests on humans that ingesting the food compounded with fenugreek gum powder lowers the level of sugar in the blood. Moreover, fenugreek seeds lower the level of cholesterol and fat in the blood and restrains biosynthesis of cholesterol in the liver. These effects are caused by galactomannan contained in albumen in seeds (fenugreek gum). Polysaccharide, or dietary fiber, generally has the effect of lowering the level of cholesterol, and fenugreek is distinctive for having the effect of lowering the level of sugar in the blood as well.

Erythritol (FIG. A-23)

Erythritol is a naturally-derived sugar substitute that looks and tastes very much like sugar, yet has almost no calories. It comes in granulated and powdered forms. It is absorbed into the bloodstream in the small intestine, before it enters the large intestine, it does not normally cause laxative effects as are often experienced after over-consumption of other sugar alcohols and most people can consume Erythritol with no side effects without causing gastric distress.

### EXAMPLE 2

### Daily Mixture B

All or some of the components of Mixture A may be combined
with some or all of the components of the following:

| | | |
|---|---|---|
| Grape Seed | Mg | 75 |
| Aloe vera | Mg | 50 |
| Orange Peel | Mg | 30 |
| Wheatgrass | G | 3.5 |
| Resveratrol/Resvida ® | Mg | 30 |
| Optional Piracetam ® | Mg | 800 |

All or some of the components from the “Other - E” group below may be combined with some or all of the components of Mixture B.

Grape Seed

Scientific Name: Vitis vinifera, V. rotundifolia, V. labrusca (FIG. B-1)

Grape seed extract contains chemicals known as polyphenols, (including the subclass of proanthocyanodins), which are recognized to be effective polyphenol antioxidants.

Grape seed extract may help to prevent and treat heart diseases such as high blood pressure and high cholesterol. By limiting oxidation, antioxidants in grape seed extract may help prevent changes, including damage to blood vessels that may contribute to the development of heart disease. Substances in grape seed extract may also block the effects of enzymes that process fats, including cholesterol from the diet. Consequently, less fat may be absorbed and more may be eliminated from the body. Other research shows that grape seed extract may help to prevent or control damage to body cells that is caused by toxins.

Aloe Vera

Scientific Name: Aloe vera (FIG. B-2)

Aloe vera is also called as Aloe barbadensis. It has a remarkable anti-viral property via a compound called acemannon. The anti-viral action of acemannon was found to extend even to transformation of the protein envelope of the HIV virus—thus preventing it from attaching to cells. However, these results are preliminary. In another study carried out by Lee Cowden at Dallas, Tex., it was seen that regular oral consumption of Aloe vera benefited several disorders of the gastrointestinal tract like irritable bowl syndrome, ulcerative colitis, esophagitis, peptic ulcer, rheumatoid arthritis, osteoarthritis, mouth lesions, sore throat, and lupus. In another study carried out by R. H. Davis at the University of Pennsylvania, it was observed that aloe vera can prevent and arrest arthritis, inhibit pain, reduce inflammation and restore bone growth.

Lee et al., at the Department of Food and Nutrition, Ho Seo University, Korea have isolated a strong antioxidative compound from a methanolic extract of Aloe barbadensis. The investigators found that the antioxidative action of the compound isolated from Aloe extract was of a comparable efficacy as that of alpha-tocopherol.

Pugh and Ross at the Department of Pharmacognosy, National Centre for Natural Products Research, University of Mississippi, have characterized a new immunostimulatory polysaccharide called Aloeride from commercial aloe vera (Aloe barbadensis). The investigators observed that Aloeride induced the expression of the mRNAs encoding IL-1beta and TNF-alpha to levels equal to those observed in cells maximally activated by LPS. According to the investigators, aloeride although just 0.015% of the aloe juice dry weight has a remarkable action on activation of macrophages and may be the single most important component of Aloe vera juice.

Orange Peel

Scientific Name: Citrus sinensis (FIG. B-3)

A class of compounds found in citrus fruit peels called polymethoxylated flavones (PMFs) have the potential to lower cholesterol more effectively than some prescription drugs, and without any side effects, according to a study by U.S. and Canadian researchers that was published in the Journal of Agricultural and Food Chemistry.

Sweet orange oil consists of about 90% d-Limonene which is known as a significant chemopreventive anti-cancer agent. The phytonutrient d-limonene is found abundantly in the peels of oranges and studies show that d-limonene may reduce the risk of some cancers.

The most important flavanone in oranges, herperidin has been shown to lower high blood pressure as well as cholesterol in animal studies, and to have strong anti-inflammatory properties. Most of the phytonutrient is found in the peel and inner white pulp of the orange, rather than in its liquid orange center.

Wheat Grass

Scientific Name: Triticum aestivum (FIG. B-4)

It is rich in Chlorophyll, Beta Carotene, Minerals & Live enzymes. An example of Nutrients: Wheat Grass of 30 grams is given below:

| Nutrient | Wheat Grass Juice | |
|---|---|---|
| Protein | 860 | mg |
| Beta carotene | 120 | IU |
| Vitamin E | 880 | mcg |
| Vitamin C | 1 | mg |
| Vitamin B₁₂ | 0.30 | mcg |
| Phosphorus | 21 | mg |
| Magnesium | 8 | mg |
| Calcium | 7.2 | mg |
| Iron | 0.66 | mg |
| Potassium | 42 | mg |

Resveratrol/Resvida® (FIG. B-5)

Resveratrol (trans-3,5,4′-trihydroxystilbene) is a substance found in the skins of certain red grapes, in peanuts, blueberries, some pines and giant knotweed. Resveratrol is produced by grapes and other plants to protect the plant against fungus, and disease. Resveratrol at low doses can retard some aspects of the aging process, including heart aging, and it may do so by mimicking some of the effects of caloric restriction, which is known to retard aging in several tissues and extend life span.

DSM Nutritional Products, a company based in Basel, Switzerland is manufacturing a pure resveratrol product called Resvida.

Piracetam® (FIG. B-6)

Piracetam® is a cyclic derivative of GABA and it is nootropic. It is a dietary supplement which is claimed to enhance cognition and memory, slow down brain aging, Alzheimer's disease, increase blood flow/oxygen to the brain, and aid stroke recovery.

### EXAMPLE 3

### Daily Mixture C

All or some of the components of Mixtures A or B may be combined
with some or all of the components of the following:

| | | |
|---|---|---|
| Gardenia | mL | 75 |
| Indian Kino Tree | Mg | 450 |
| Guduchi | Mg | 250 |
| Gurmar | Mg | 400 |
| Cinnamon | Mg | 750 |
| Fenugreek | G | 2.5 |
| Clove | Mg | 100 |
| Fennel | Mg | 450 |

All or some of the components from the “Other - E” group below may be combined with some or all of the components of Mixture C.

Gardenia

Scientific Name: Gardenia jasminoides (FIG. C-1)

Gardenia fruit extract which may be used in to treat symptoms of Type-2 diabetes and contains a chemical that reverses some of the pancreatic dysfunctions that underlie the disease.

The primary active components of gardenia are iridoid glycosides (mainly geniposide and gardenoside), chlorogenic acid, and ursolic acid. Gardenia extract contains the chemical called “genipin.” This Genipin stimulates the secretion of insulin in control but not UCP2-deficient pancreas. Acute addition of genipin to isolated pancreatic tissue reversed high glucose and obesity induced dysfunction of insulin-producing beta cells. A derivative of genipin that lacked the chemical's cross-linking activity continued to inhibit UCP2.

Indian Kino Tree

Scientific Name: Pterocarpus Marsupium (FIG. C-2)

Common Names: Indian Kino Tree, Malabar Kino Tree, Kino

Pterocarpus marsupium is an anti-diabetic plant indigenous to South India. Mechanisms such as the stimulating or regenerating effect on beta cells or extrapancreatic effects are proposed for the hypoglycemic action of Indian Kino Tree extract. The isoflavone from Pterocarpus marsupium may activate glucose transport, improving glucose uptake in the body and is quite similar to insulin in its action. Administration of Pterocarpus marsupium led to decrease in blood glucose levels by 38% and 60% on the 15th and 30th day of the experiment.

Guduchi

Scientific Name: Tinospora cordifolia (FIG. C-3)

Guduchi is bitter, pungent and astringent in taste, sweet in the post digestive effect, hot in potency and has a special potency as an anti-toxin. Oral administration of the extract of Tinospora cordifolia roots for 6 weeks resulted in a significant reduction in blood and urine glucose and in lipids in serum and tissues in alloxan diabetic rats.

Gurmar/Periploca of the Woods

Scientific Name: Gymnema sylvestre (FIG. C-4)

It may be used in an all natural medication for diabetes with other ingredients such as cinnamon, chromium, zinc, biotin, banaba, huckleberry and bitter melon. Extracts of Gymnema is used for treatment of hyperglycemia, obesity, high cholesterol levels, anemia and digestion. The active ingredient is thought to be gurmenic acid which is useful against obesity. Gymnemic acids delay the glucose absorption in the blood.

When Gymnema leaf extract is administered to a diabetic patient, there is stimulation of the pancreas by virtue of an increase in insulin release. There are some possible mechanisms by which the leaves and especially Gymnemic acids from Gymnema sylvestre exert its hypoglycemic effects are:
It increases secretion of insulin.
It promotes regeneration of islet cells.
It increases utilization of glucose: it is shown to increase the activities of enzymes responsible for utilization of glucose by insulin-dependant pathways, an increase in phosphorylase activity, decrease in gluconeogenic enzymes and sorbitol dehydrogenase.
It causes inhibition of glucose absorption from the intestine.

The leaves are also noted for lowering serum cholesterol and triglycerides.

Cinnamon

Scientific Name: Cinnamomum zeylanicum, Cinnamomum cassia blume (FIG. C-5)

Cinnamon's flavor is due to an aromatic essential oil which makes up 0.5% to 1% of its composition. Chemical components of the essential oil include ethyl cinnamate, eugenol, cinnamaldehyde, beta-caryophyllene, linalool and methyl chavicol. Recent research has revealed that constituents in cinnamon bark called procyanidin Type-A polymers help maintain the body's ability to metabolize glucose in a healthy way. Cinnamon also contains Calcium, Iron, Vitamins and Fiber.

Fenugreek

Scientific Name: Trigonella foenum-graecum (FIG. C-6)

Fenugreek is used both as an herb (the leaves) and as a spice (the seed). Supplements of fenugreek seeds were shown to lower serum cholesterol, triglyceride, and low-density lipoprotein in human patients and experimental models of hypercholesterolemia and hypertriglyceridemia. Several human trials have demonstrated the anti-diabetic effect of its seeds. It is often cited as a natural remedy for migraines, blood pressure and LDL blood cholesterol levels.

Clove

Scientific Name: Syzygium aromaticum (FIG. C-7)

Extracts of cloves were found to improve the function of insulin and to lower glucose, total cholesterol, LDL and triglycerides in people with Type-2 diabetes.

Clove extract acts like insulin in hepatocytes and hepatoma cells by reducing phosphoenolpyruvate carboxykinase (PEPCK) and glucose 6-phosphatase (G6Pase) gene expression. Much like insulin, clove-mediated repression is reversed by PI3K inhibitors and N-acetylcysteine (NAC).

Fennel

Scientific Name: Foeniculum vulgare (FIG. C-8)

Fennel contains anethole, which acts as phytoestrogens—an herbal composition for the treatment of diabetes.

### EXAMPLE 4

### Daily Mixture D

All or some of the components of Mixtures A, B, or C may be
combined with some or all of the components of the following:

| | | |
|---|---|---|
| Decaffeinated Green Tea | mL | 50 |
| Coccinia indica | Mg | 250-1000 |
| Salacia oblonga | Mg | 250-1000 |
| Resistance Starch | G | 3.5 |
| Diachrome ™ | Mcg | 500 |
| InsuVital ™ | Mg | 5 |
| Pycnogenol ® | Mg | 50 |
| Biotin | Mcg | 5-30 |
| Beta-Glucan | Mg | 30-500 |
| Conjugated Linoleum Acid (CLA) | G | 3 |
| Alpha-Lipoic Acid (ALA) | Mg | 600 |
| SX-Fraction | Mg | 70 |

All or some of the components from the “Other - E” group may be combined with some or all of the components of Mixture D.

For pH balance Citric, Malic, Fumaric, and Tartaric acid(s) may be added. Also preservatives such as Sodium Benzoate, Potassium Sorbate and/or Disodium EDTA may be added.

Decaffeinated Green Tea

Scientific Name: Camellia sinensis (FIG. D-1)

Green tea polyphenols have been found to be potent inhibitors of amylase. Just one cup of green tea was found to inhibit 87% of amylase's activity. And if less sugar gets into the bloodstream, blood glucose levels will automatically be lowered

Ivy Gourd

Scientific Name: Coccinia indica (FIG. D-2)

Coccinia indica may reduce blood sugar levels by about 20 percent. A one gram daily dose of Coccinia indica extract led to an 18% reduction in blood sugar levels after meals, according to a double blind, placebo controlled, randomized study.

Salacia

Scientific Name: Salacia oblonga (FIG. D-3)

Salacia oblonga contains two potent a-Glucosidase inhibitors: Salicinol and Kotalanol 9. Salacia oblonga has also been found to show inhibitory activity on Aldose Reductase which is related to diabetic complications as peripheral neuropathy, retinopathy, and cataracts. Salacia oblonga is an effective anti-diabetic and dieting agent. 2.5 to 5.0 grams of Salacia oblonga daily is effective in lowering the blood glucose, serum cholesterol, triglycerides and increasing the HDL cholesterol levels of Type-2 diabetes

Resistant Starch (FIG. D-4)

Resistant starch has been categorized into four types:
Physically inaccessible or digestible resistant starch, such as that found in seeds or legumes and unprocessed whole grains
Resistant starch that occurs in its natural granular form, such as uncooked potato, green banana flour and high amylose corn
Resistant starch that is formed when starch-containing foods are cooked and cooled such as in bread, cornflakes and cooked-and-chilled potatoes or retrograded high amylose corn
Starches that have been chemically modified to resist digestion. This type of resistant starches can have a wide variety of structures and are not found in nature

Resistant starch is a starch that escapes digestion in the small intestine of healthy individuals. Resistant starch is considered the third type of dietary fiber, as it can deliver some of the benefits of insoluble fiber and some of the benefits of soluble fiber.

Some carbohydrates, such as sugars and most starch are rapidly digested and absorbed as glucose into the body through the small intestine and subsequently used for short-term energy needs or stored. Resistant starch, on the other hand, resists digestion and passes through to the large intestine where it acts like dietary fiber.

Diachrome™ (FIG. D-5)

Diachrome™ (patented composition of chromium picolinate and biotin). It not only improves blood sugar levels, but decreases LDL cholesterol which will help people with diabetes in lowering LDL cholesterol.”

InsuVital™ (FIG. D-6)

InsuVital™ consists of extensively hydrolysed casein, a protein which is present in milk. A proprietary enzyme has been used to cut (“hydrolyse”) the casein into smaller pieces, also called peptides. The mixture of peptides of 2 or 3 amino acids forms the active part of InsuVital™. These peptides can stimulate the secretion of insulin from the pancreas, even in people suffering from Type-2 diabetes.

Pycnogenol® (FIG. D-7)

Pycnogenol® Powder provides significant health protection in diabetes as demonstrated in controlled clinical trials which caused the lowering of glucose levels and improvements in microvascular health problems such as diabetic microangiopathy, foot ulcer healing, muscle cramps, prevention and improvement of diabetic retinopathy.

Biotin (FIG. D-8)

Biotin, also known as vitamin H or B7, has the chemical formula C10H16N2O3S. People with type 2 diabetes often have low levels of biotin. Biotin may be involved in the synthesis and release of insulin. Preliminary studies in both animals and people suggest that biotin may help improve blood glucose control in those with diabetes, particularly Type-2 diabetes. Specifically, biotin doses in excess of nutritional requirements lower postprandial glucose and improve glucose tolerance.

Beta-Glucan (FIG. D-9)

WGP Beta Glucan stimulates the immune system by activating the macrophage cells, powerful, immune cells that engulf foreign invaders and alert the body's other defenses. By increasing the activity of the macrophage cells, the immune system is thereby made stronger and better able to fight off foreign invaders and health challenges.

Conjugated Linoleum Acid (FIG. D-10)

Conjugated Linoleum Acid (CLA) may protect against development of diabetes, atherosclerosis, chronic inflammation and colon cancer. It is found predominantly in dairy products such as milk, cheese and meat, and are formed by bacteria in ruminants that take linoleic acids—fatty acids from plants—and convert them into Conjugated Linoleum Acid.

Alpha-Lipoic Acid (FIG. D-11)

Alpha lipoic acid (ALA) is a vitamin-like substance that helps to make energy in your body. As an antioxidant, it is used to treat acquired immunodeficiency syndrome (AIDS).

Several studies suggest that treatment with alpha-lipoic acid may help reduce pain, burning, itching, tingling, and numbness in people who have nerve damage (called peripheral neuropathy) caused by diabetes. Alpha-lipoic acid speeds the removal of glucose (sugar) from the blood of people with diabetes and that this antioxidant may prevent kidney damage associated with diabetes in animals.

Because alpha-lipoic acid can pass easily into the brain, it has protective effects on brain and nerve tissue and shows promise as a treatment for stroke and other brain disorders involving free radical damage. Animals treated with alpha-lipoic acid, for example, suffered less brain damage and had a four times greater survival rate after a stroke than the animals who did not receive this supplement. While animal studies are encouraging, more research is needed to understand whether this benefit applies to people as well.

Seed oils are the richest sources of alpha-lipoic acid, notably those of rapeseed (canola), soybeans, walnuts, flaxseed (Linseed), perilla, chia and hemp. Alpha-lipoic acid is also obtained from the thylakoid membranes of the green leaves of broadleaf plants (the membranes responsible for photosynthesis). Chia (Salvia hispanica), Kiwi (Actinidia chinensis), Perilla (Perilla frutescens), Flax (Linum usitatissimum), Lingonberry (Vaccinium vitis-idaea), Purslane (Linum usitatissimum), Seaberry (Hippophae rhamnoides) has 64%, 62%, 58%, 55%, 49%, 35% and 32% alpha-lipoic acid respectively.

SX-Fraction (FIG. D-12)

Maitake SX fraction is safe and is very helpful for Type-2 diabetes. Studies on SX-Fraction have been conducted at Georgetown University and New York Medical College respectively over the last two years. The results show that SX-Fraction does indeed possess a more potent ability to enhance insulin sensitivity for controlling blood sugar levels and lowering blood pressure than X-fraction.

Other—E

The following ingredients can be included in any of mixtures A, B, C and D above.

Stevia

Scientific Name: Stevia rebaudiana (FIG. E-1)

Common Names: Sweet Leaf, Sugar Leaf, Sweet Honey Leaf, Rebiana. Stevia (also known as Eupatorium rebaudianum Bertoni) is known simply as Stevia or Sutebia or Satiwia.

Stevia is an all-natural sweetener that contains no chemicals and is 250 times sweeter than sugar but has none of the calories, and it can be used as sugar. Rather than raise blood sugar like most other natural sweeteners, Stevia actually lowers it. Stevia helps control blood glucose and promotes insulin creation. As a sugar substitute, it has a slower onset/longer duration than that of sugar. Results of the study led the researchers at the time to conclude that the plant extract Stevioside may potentially be used as a new medication for Type-2 diabetes.

Loquat

Scientific Name: Eriobotrya japonica (FIG. E-2)

Loquat leaves contain corosolic acid, clinically proven to activate facilitative glucose transporters within the cell to utilize glucose more efficiently. Corosolic acid is clinically proven to activate cell glucose-transporter “shuttles” and thus helps balance blood glucose levels. Corosolic acid shows a memory effect of blood glucose lowering even after the treatment is stopped.

Cabbage

Scientific Name: Brassica oleracea (FIG. E-3)

A recent study reveals the anti-diabetic effects of cabbage. This is as effective as high doses of Insulin. It is a low calorie, anti-cancer, fiber filled food that lowers excess sugar naturally.

Chongcao

Scientific Name: Cordyceps sinensis (FIG. E-4)

Common Names: Cordyceps Mushroom, Caterpillar Fungus, Chongcao, Deer Fungus Parasite, Dong Chong Xia Cao, Chong Cao.

Chongcao is helpful in reducing high cholesterol and improving male function. It fights against stress, controls blood sugar levels, lowers blood pressure and reduces cancer risk

Indian Gooseberry

Scientific Name: Phyllanthus emblica (Also known as Emblica officinalis) (FIG. E-5)

Indian gooseberry has undergone preliminary research, demonstrating in vitro antiviral and antimicrobial properties. Experimental preparations of leaves bark or fruit have shown potential efficacy against cancer, age-related renal disease, and diabetes. A human pilot study demonstrated reduction of blood cholesterol levels in both normal and hypercholesterolemic men.

Onion Family (Onion, Garlic, Scallion and Leeks)

Scientific classification: Liliaceae Family (FIG. E-6)

Two sets of compounds make up the majority of onion's known active constituents—sulfur compounds, such as allyl propyl disulphide (APDS), and flavonoids, such as quercetin. Each of these groups of compounds has multiple medicinal actions. APDS has been shown to block the breakdown of insulin by the liver and possibly to stimulate insulin production by the pancreas, thus increasing the amount of insulin and reducing sugar levels in the blood.

Garlic is also alleged to help regulate blood sugar levels. Regular and prolonged use of therapeutic amounts of aged garlic extracts lower blood homocysteine levels, and has shown to prevent some complications of diabetes mellitus.

Guar Gum

Scientific Name: Cyamopsis tetragonolobus (FIG. E-7)

It refers to a guaran or galactomannan. It is primarily the ground endosperm of guar beans. The guar seeds are dehusked, milled and screened to obtain the guar gum.

Guar gum is of interest with regards to both weight loss and diabetic diets. These decreases are thought to be a function of its high soluble fiber content. Guar gum was found to improve dietary glucose tolerance. Research has revealed the water soluble fiber in guar gum may help people with diabetes by binding with glucose in the gastrointestinal tract, thus preventing its absorption. Guar gum gives a sense of fullness after eating therefore it is good for obesity.

Licorice Weed

Scientific Name: Scoparia dulcis (Synonyms: Scoparia grandiflora, Scoparia ternata, Capraria dulcis, Gratiola micrantha) (FIG. E-8)

Also known as: Vassourinha, ñuñco pichana, anisillo, bitterbroom, boroemia, broomweed, brum sirpi, escobilla, mastuerzo, piqui pichana, pottipooli, sweet broom, tapixava and tupixaba.

Licorice weed contains the phytochemicals flavone and terpene, which are responsible for its biological properties. Methanol extract of vassourinha leaves also showed toxic actions against cancer cells (with a 66% inhibition rate).

Ranawara or Avaram

Scientific Name: Cassia auriculata (FIG. E-9)

This plant is said to contain a cardiac glucoside (sennapicrin) and sap, leaves and bark yield contain oxymethylanthraqunone while the latter contains tannins. The root is used against fevers, diabetes, diseases of the urinary system and constipation.

Phyllanthus Plant

Scientific Name: Phyllanthus niruri (FIG. E-10)

Common Names: Phyllanthus Plant, Child Pick-a-back, Gulf Leafflower, Black Catnip, Meniran, Chanca Piedra, Shatterstone, Stone Breaker, Quebra Pedra, Gale Of Wind, Carry Me Seed, Creole Senna, Daun Marisan.

Phyllanthus Plant helps prevent jaundice, diabetes, dyspepsia, ulcers, sores, swellings, ophthalmia and chronic dysentery.

Indian Gentian

Scientific Name: Swertia chirata (FIG. E-11)

Common Names: Clearing Nut Tree, Bitter Stick, Chirette Indian, Dowa I Pechish, Indian Gentian. Indian gentian contains xanthones and amarogentin, a glycoside.

The ethanolic extract of Swertia chirayita exhibits hypoglycemic activity. The hexane fraction containing swerchirin, the main hypoglycemic principle, induced a significant fall in blood sugar in albino rats. The compound may have clinical applications in the control of diabetes

Indian Beech

Scientific Name: Pongamia pinnata (FIG. E-12)

Common Names: Indian Beech, Poongam Oil Tree, Honge, Ponge. Flowers are useful in diabetes. Oral administration of ethanolic extract of Pongamia pinnata flower shows significant anti-hyperglycemic, anti-lipid per-oxidative effect and enhancement in antioxidant defense.

Tulsi

Scientific Name: Ocimum sanctum, Ocimum tenuiflorum (FIG. E-13)

Tulsi is helpful in a significant reduction in total cholesterol levels and blood glucose levels due to its antioxidant properties.

Bael

Scientific Name: Aegle marmelos (FIG. E-14)

Common Names: Bael, Quince, Apple Wood, Holy Fruit Tree

Leaves are scientifically proved to be anti-diabetic. Drinking fresh juice of leaves daily along with pinch of black pepper will take care of some excess body sugar.

Leaves of Custard Apple

Scientific Name: Annona reticulate (FIG. E-15)

Recent study reveals that newly emerged leaves of long pepper lowers blood sugar levels effectively.

Any Ingredient Such As Solid, Liquid, Vapor, Extract, Compound, Derivative, Bioactive Component(s) From Any Part Of; A Seed, Root, Leaf, Bark, Plant, Fruit And/Or Vegetable Consisting Of At Least One Of The Following:

Acerola Berry, Agave, Algae (Spirulina), Allium Family (Onions, Garlic, Chives, Leeks, Shallots and Scallions), Amla, Apple, Apricot, Araca-Boi, Aronia, Ashwagandha (Withania somnifera), Avocado, Banana, Bergamot, Bilberry, Bitter Yam (Dioscorea bulbifera), Blackberry, Black Elder, Black Johannisbeerry, Boysenberry, Broccoli, Caja, Camu-Camu, Cantaloupe, Carrot, Cashew, Cherry, Cocoa, Coconut Milk, Coconut Water, Coffee (Coffea Arabica), Coffeeberry, Cranberry, Crenshaw, Cucumber, Cupuacu, Dragon Fruit, Elderberry, Fig (Ficus glomerata), Gac (Momordica cochinchinensis), Ginger, Ginseng (Panax Ginseng), Graviola, Grape, Greenbean, Guarana, Guava, Honeydew, Indian Kudzu (Pueraria tuberosa), Kiwi, Lemon, Loganberry, Lulo, Maitake Mushroom (Grifola frondosa), Marking Nut Tree (Salacia reticulata), Mango, Muskmelon, Nashi, Nectarine, Neem (Azadirachta indica), Noni, Olive, Orange, Papaya, Passion Flower, Passion Fruit, Peach, Pear, Pepper, Persimmon, Pineapple, Plum, Prune, Pumpkin, Raspberry, Spinach, Sprout, Soy, Star Fruit, Strawberry, Sweet Potato (Ipomoea batatas), Sun Flower, Tangerine, Tarragon, Tea (Camellia sinensis), Tomato, Vitaberry, Water, Watermelon, Winter Cherry, Wine, Yohimbe, Yumberry & Others.

Method Of Preparation:

The ingredients of Mixtures A, B, C, D, E and others may be processed or heated at ultra-high temperature at about 250 degree centigrade for about 5 seconds under any food-grade inert gas(s) such as nitrogen and/or argon or any combination thereof to reduce microbial contamination, nutritional degradation and oxidation. Moreover, the human body may not absorb many ingredients easily. Ingredients such as Turmaric and/or Curcumin may be encapsulated in a bio-compatible nano-sphere having many nano-holes such that ingredients can diffuse into the blood slowly over time.

Dendrimers and cyclodextrins are like microscopic balls with a dense network of branches. Dendrimers and cyclodextrins have an incredibly high surface area to volume ratio and if they are bio-compatible and non-toxic, they may be used to diffuse nutrients into the blood slowly over time. Similar encapsulation method applies to flavor and color as well to preserve their effects over time.

FIG. F-1 shows a picture of cyclodextrins. A cyclodextrin is a circular sugar ring molecule. Once water is added to cyclodextrin, cyclodextrin forms a cavity that can hold insoluble guest molecules. Cyclodextrin can act like a vessel and helps to solublize insoluble ingredient. Once guest molecules go into solution, these particles spread out and enhance bioavailability by significantly enhancing the surface area.

Alternatively, the protein Casein (80% protein content of cow's milk) can naturally form of spherical micelles with diameters ranging from 50 to 500 nanometers. The stability of these micelles during thermal processing makes them a suitable as nano-encapsulator for insoluble ingredients.

FIG. F-2 shows a method of encapsulation of an ingredient 100 within a glycolipid nanotube (120) in water solution (140).

The above detailed description is provided to illustrate specific embodiments of the present invention and is not intended to be limiting. Numerous variations and modification within the scope of the present invention are possible.

The present invention is set forth in the accompanying claims.

1. A mixture containing two or more ingredients in the form of a solid, a liquid, a vapor, an extract, a compound, a derivative, a bioactive molecule(s) and a bioactive component(s) derived from any part of a seed, a root, a leaf, a bark, a plant, a fruit and a vegetable and selected from the group consisting of: Algae (Spirulina), Euterpe oleracea, Lycium barbarum, Hippophae rhamnoides, Garcinia mangostana, Ribes spp, Daucus carota, Vitis vinifera, Vitis labrusca, Vitis rotundifolia, Punica granatum, Vaccinium spp, Vaccinium angustifolium, Vaccinium boreale, Vaccinium caesariense, Prunus cerasus, Momordica charantia, Momordica chinensis, Momordica elegans, Momordica indica, Momordica operculata, Momordica sinensis, Sicyosfauriei, Momordica cochinchinensis, Syzygium cumini, Curcuma longa, Tetrahydrocurcuminoid, Curcumin's GO-Y030 bioactive molecule, Curcumin's GO-Y031 bioactive molecule, Garcinia cambogia, Fenugreek Gum, Aloe vera, Citrus sinensis Peel, Triticum aestivum, Gardenia jasminoides, Pterocarpus marsupium, Tinospora cordifolia, Gymnema sylvestre, Cinnamomum zeylanicum, Cinnamomum cassia blume, Trigonella foenum-graecum, Syzygium aromaticum, Foeniculum vulgare, Camellia sinensis, Coccinia indica, Salacia oblonga, Cyamopsis tetragonolobus, Stevia rebaudiana, Eriobotrya japonica, Brassica oleracea, Cordycepss sinensis, Phyllanthus emblica, Liliaceae Family (Allium cepa, Allium sativum), Scoparia dulcis, Cassia auriculata, Phyllanthus niruri, Swertia chirata, Pongamia pinnata, Ocimum sanctum, Ocimum tenuiflorum, Aegle marmelos, Annona reticulate, Salacia reticulata, Grifola frondosa, Ipomoea batatas, Withania somnifera, Pueraria tuberosa, Dioscorea bulbifera, Panax ginseng, Ficus glomerata, Synsepalum dulcificum, Morus alba, Morus rubra, Morus nigra, Malus domestica and Azadirachta indica.

2. The mixture of claim 1, further comprising one or more additional ingredients selected from the group consisting of: Proteins, Secoisolariciresinol Diglycoside (SDG), Salvia hispanica, Linum usitatissimum, resistant starch, fibers, phytosterols, vitamins, omega family of fatty acids, mineral ions, natural enzymes, designer enzymes, Super Citrimax®, Hydroxy Citric Acid (HCA), L-Carnitine, Coenzyme Q₁₀, Resveratrol, Resvida®, Piracetam®, Diachrome®, InsuVital®, Pycnogenol®, Biotin, Beta-Glucan, Conjugated Linoleum Acid (CLA), Alpha-Lipoic Acid (ALA) and SX-Fraction.

3. The mixture of claim 1, further comprising one or more additional ingredients selected from the group consisting of: an edible solid(s), an edible liquid(s), an edible color(s), an edible stabilizer(s), an edible flavor(s) and an edible preservative(s).

4. The mixture of claim 2, further comprising one or more additional ingredients selected from the group consisting of: an edible solid(s), an edible liquid(s), an edible color(s), an edible stabilizer(s), an edible flavor(s) and an edible preservative(s)

5. The mixture of claim 1, wherein the mixture is provided in a form suitable for use as a food, a drink, a supplement, a drug, a cosmetic and a hygienic product.

6. The mixture of claim 2, wherein the mixture is provided in a form suitable for use as a food, a drink, a supplement, a drug, a cosmetic and a hygienic product.

7. The mixture of claim 3, wherein the mixture is provided in a form suitable for use as a food, a drink, a supplement, a drug, a cosmetic and a hygienic product.

8. The mixture of claim 4, wherein the mixture is provided in a form suitable for use as a food, a drink, a supplement, a drug, a cosmetic and a hygienic product.

9. A mixture comprising:
a. One or more ingredients selected from the group consisting of: proteins, Secoisolariciresinol Diglycoside (SDG), Salvia hispanica, Linum usitatissimum, resistant starch, fibers, phytosterols vitamins, omega family of fatty acids, mineral ions, natural enzymes, designer enzymes, Super Citrimax®, Hydroxy Citric Acid (HCA), L-Carnitine, Coenzyme Q₁₀, Resveratrol, Resvida®, Piracetam®, Diachrome®, InsuVital®, Pycnogenol®, Biotin, Beta-Glucan, Conjugated Linoleum Acid (CLA), Alpha-Lipoic Acid (ALA) and SX-Fraction; and
b. A product, which is selected from the group consisting of: a food, a drink, a supplement, a drug, a cosmetic and a hygienic product.

10. A mixture including ingredients in the form a solid, a liquid, a vapor, an extract, a compound, a derivative, a bioactive molecule(s) and a bioactive component(s) derived from any part of a seed, a root, a leaf, a bark, a plant, a fruit and a vegetable comprising Spirulina.

11. A mixture including ingredients in the form a solid, a liquid, a vapor, an extract, a compound, a derivative, a bioactive molecule(s) and a bioactive component(s) derived from any part of a seed, a root, a leaf, a bark, a plant, a fruit and a vegetable comprising Triticum aestivum.

12. A mixture including ingredients in the form a solid, a liquid, a vapor, an extract, a compound, a derivative, a bioactive molecule(s) and a bioactive component(s) derived from any part of a seed, a root, a leaf, a bark, a plant, a fruit and a vegetable comprising Momordica charantia.

13. A mixture including ingredients in the form a solid, a liquid, a vapor, an extract, a compound, a derivative, a bioactive molecule(s) and a bioactive component(s) derived from any part of a seed, a root, a leaf, a bark, a plant, a fruit and a vegetable comprising Momordica cochinchinensis.

14. A mixture including ingredients in the form a solid, a liquid, a vapor, an extract, a compound, a derivative, a bioactive molecule(s) and a bioactive component(s) derived from any part of a seed, a root, a leaf, a bark, a plant, a fruit and a vegetable comprising Syzygium cumini.

15. A mixture including ingredients in the form a solid, a liquid, a vapor, an extract, a compound, a derivative, a bioactive molecule(s) and a bioactive component(s) derived from any part of a seed, a root, a leaf, a bark, a plant, a fruit and a vegetable comprising Coccinia indica.

16. A mixture including ingredients in the form a solid, a liquid, a vapor, an extract, a compound, a derivative, a bioactive molecule(s) and a bioactive component(s) derived from any part of a seed, a root, a leaf a bark, a plant, a fruit and a vegetable and comprising Salacia oblonga.

17. A mixture including ingredients in the form a solid, a liquid, a vapor, an extract, a compound, a derivative, a bioactive molecule(s) and a bioactive component(s) derived from any part of a seed, a root, a leaf, a bark, a plant, a fruit and a vegetable comprising Curcuma longa.

18. A method of preparing a water-soluble or an oil-soluble mixture, comprising mixing two or more of the ingredients selected from the group consisting of: a Turmeric, a Turmeric derivative(s), a Turmeric bioactive molecule(s), a Curcumin, a Curcumin derivative(s) and a Curcumin bioactive molecule(s).

19. A mixture as in claim 10 wherein the ingredients are encapsulated in a nano-vessel.

20. A mixture as in claim 11 wherein the ingredients are encapsulated in a nano-vessel.

21. A mixture as in claim 12 wherein the ingredients are encapsulated in a nano-vessel.

22. A mixture as in claim 13 wherein the ingredients are encapsulated in a nano-vessel.

23. A mixture as in claim 14 wherein the ingredients are encapsulated in a nano-vessel.

24. A mixture as in claim 15 wherein the ingredients are encapsulated in a nano-vessel.

25. A mixture as in claim 16 wherein the ingredients are encapsulated in a nano-vessel.

26. A mixture as in claim 17 wherein the ingredients are encapsulated in a nano-vessel.

27. A method of preparation of a food, a drink, a supplement, a drug, a cosmetic and a hygienic product comprising thermally processing the product in a vessel under a food-grade inert gas.

28. A method as in claim 27, wherein the food-grade inert gas includes at least one of following: a nitrogen and an argon.

29. The method of claim 27, wherein the vessel is a packaging for the product.

30. The method of claim 27, wherein the vessel is a packaging for a color of the product.

31. The method of claim 27, wherein the vessel is a packaging for a flavor of the product.

32. A method for preparing a food, a drink, a supplement, a drug, a cosmetic and a hygienic product comprising mixing two or more ingredients in the form of a solid, a liquid, a vapor, an extract, a compound, a derivative, a bioactive molecule(s) and a bioactive component(s) from any part of a seed, a root, a leaf, a bark, a plant, a fruit and a vegetable and selected from the group consisting of: Algae (Spirulina), Euterpe oleracea, Lycium barbarum, Hippophae rhamnoides, Garcinia mangostana, Ribes spp, Daucus carota, Vitis vinifera, Vitis labrusca, Vitis rotundifolia, Punica granatum, Vaccinium spp, Vaccinium angustifolium, Vaccinium boreale, Vaccinium caesariense, Prunus cerasus, Momordica charantia, Momordica chinensis, Momordica elegans, Momordica indica, Momordica operculata, Momordica sinensis, Sicyosfauriei Momordica cochinchinensis, Syzygium cumini, Curcuma longa, Tetrahydrocurcuminoid, Curcumin's GO-Y030 bioactive molecule, Curcumin's GO-Y031 bioactive molecule, Garcinia cambogia, Fenugreek Gum, Aloe vera, Citrus sinensis Peel, Triticum aestivum, Gardenia jasminoides, Pterocarpus marsupium, Tinospora cordifolia, Gymnema sylvestre, Cinnamomum zeylanicum, Cinnamomum cassia blume, Trigonella foenum-graecum, Syzygium aromaticum, Foeniculum vulgare, Camellia sinensis, Coccinia indica, Salacia oblonga, Cyamopsis tetragonolobus, Stevia rebaudiana, Eriobotrya japonica, Brassica oleracea, Cordycepss sinensis, Phyllanthus emblica, Liliaceae Family (Allium cepa, Allium sativum), Scoparia dulcis, Cassia auriculata, Phyllanthus niruri, Swertia chirata, Pongamia pinnata, Ocimum sanctum, Ocimum tenuiflorum, Aegle marmelos, Annona reticulate, Salacia reticulata, Grifola frondosa, Ipomoea batatas, Withania somnifera, Pueraria tuberosa, Dioscorea bulbifera, Panax ginseng, Ficus glomerata. Synsepalum dulcificum, Morus alba, Morus rubra, Morus nigra, Malus domestica and Azadirachta indica

33. The method of claim 32, further comprising one or more additional ingredients selected from the group consisting of: Proteins, Secoisolariciresinol Diglycoside (SDG), Salvia hispanica, Linum usitatissimum, resistant starch, fibers, phytosterols, vitamins, omega family of fatty acids, mineral ions, natural enzymes, designer enzymes, Super Citrimax®, Hydroxy Citric Acid (HCA), L-Carnitine, Coenzyme Q₁₀, Resveratrol, Resvida®, Piracetam®, Diachrome®, InsuVital®, Pycnogenol®, Biotin, Beta-Glucan, Conjugated Linoleum Acid (CLA), Alpha-Lipoic Acid (ALA) and SX-Fraction.

34. The method of claim 32, further comprising one or more additional ingredients selected from the group consisting of: an edible solid(s), an edible liquid(s), an edible color(s), an edible stabilizer(s), an edible flavor(s) and an edible preservative(s).

35. The method of claim 33, further comprising one or more additional ingredients selected from the group consisting of: an edible solid(s), an edible liquid(s), an edible color(s), an edible stabilizer(s), an edible flavor(s) and an edible preservative(s).
